(19) United States
(12) Patent Application Publication
(10) Pub. No.: US 2024/0207149 A1
(43) Pub. Date: Jun. 27, 2024

(54) Method For Cleaning Surfaces And Treatment Solution For Same

(71) Applicant: epitome GmbH, Wien (AT)

(72) Inventors: Wilhelm Zackl, Wien (AT); Christian Feichter, Wien (AT); Jan Hasenbichler, Arnoldstein (AT); Paul Hoffmann, Wien (AT)

(73) Assignee: epitome GmbH, Wien (AT)

(21) Appl. No.: 18/531,322

(22) Filed: Dec. 6, 2023

(30) Foreign Application Priority Data
Dec. 22, 2022 (DE) 10 2022 134 602.1

(51) Int. Cl. A61K 8/22; A61K 8/19; A61Q 11/00

(52) U.S. Cl. CPC A61K 8/22; A61K 8/19; A61Q 11/00; A61K 2800/621; A61K 2800/622; A61K 2800/651

(57) ABSTRACT

A method for cleaning surfaces, wherein the surface to be cleaned is simultaneously or successively brought into contact with two components of a treatment solution, wherein a first component of the treatment solution has a first active ingredient that is embodied to couple to a contaminant to be cleaned off and is embodied to stimulate or catalyze a chemical reaction of a second active ingredient in a second component of the treatment solution, wherein the second active ingredient used in the second component of the treatment solution is a gas mixture having at least two gaseous components that react chemically with each other under the influence of the first active ingredient.

### RELATED APPLICATIONS

This patent application claims priority under 35 U.S.C. § 119 based on German Patent Application DE 10 2022 134 602.1, filed on Dec. 22, 2022, the disclosure of which is incorporated herein by reference.

### FIELD OF THE INVENTION

The invention relates to a method for cleaning surfaces, a treatment solution for same, and the use thereof in an oral cavity.

### BACKGROUND OF THE INVENTION

In the field of tooth cleaning, there is a continuing quest for improvements in cleaning. Conventional cleaning with a toothbrush and toothpaste has multiple disadvantages.

Conventional toothpastes have up to 20% abrasive components; these abrasive components together with too much pressure exerted on the brush by the user can lead to excessive removal of tooth material over time. With the increasing average life expectancy, this now leads to the fact that by old age, teeth have been broken down by cleaning and problems arise as a result.

The toothbrush also has the disadvantage that it can damage the gums, especially with incorrect brushing so that periodontal disease is a common problem. The majority of the biofilm that constitutes the contamination is located directly above and below the gums, which is why cleaning at or next to the gums is very important. This is particularly difficult for toothbrushes because the brush comes into contact with the gums and irritates them.

In addition, brushing with a toothbrush and toothpaste is not sufficient from an oral hygiene standpoint since the interdental spaces in particular (up to 40% of the surface to be cleaned) and the gingival pockets are not cleaned sufficiently because the toothbrush does not reach these areas.

Plaque (=oral biofilm), which is formed by bacterial processes and from which tartar forms in later stages, is a contaminating film that is comparatively sticky and also sticks to itself well and cannot be easily removed, even when cleaned in direct contact with the toothbrush, especially not in the interdental spaces, into which the toothbrush can only penetrate to a limited degree if at all.

Conventional cleaning with a toothbrush therefore requires additional cleaning measures, for example the use of dental floss or interdental brushes, in order to clean in the interdental spaces, especially the areas where the teeth are contact each other, but also the interdental spaces. Even when dental floss is used, however, there is still a certain possibility of incorrect use because in particular the gums can also be injured with dental floss, particularly in the region of the interdental pockets where the bacterial load is especially high. This can lead to gingivitis, among other things.

In the past, a variety of attempts have been made to embody cleaning in other ways. For example, it is also known to clean the interdental spaces with water jet devices. In this context, it has turned out that the water jet devices of earlier times were in fact able to achieve cleaning, but the hardness of the jet could easily damage the gums. Modern devices have been significantly reduced in terms of the jet power so that they no longer directly cause damage to the gums, but the cleaning performance has also become so poor as to render these devices largely ineffective.

There have also been many attempts to provide so-called ultrasonic brushes, in which a vibration of the toothbrush, which is used for cleaning and ultimately, together with toothpaste, in turn achieves an abrasive cleaning, is superimposed with ultrasonic vibrations, which supposedly achieve a cleaning effect. It has turned out, though, that such toothbrushes are not capable of coupling the ultrasound into the oral cavity in such a way that a cleaning effect would even be detectable. Such so-called ultrasonic toothbrushes are therefore not significantly better than a conventional manual toothbrush.

Other electric toothbrushes in which the brush head makes circular or vibrating movements often do have pressure control, but ultimately these movements also lead to abrasive brushing.

In the field of contactless cleaning, the cleaning effect of imploding or collapsing vapor bubbles has been discussed in recent years. Such vapor bubbles have been obtained either through the application of ultrasound, through spot heating by laser, or through hydrodynamic cavitation. When the vapor bubbles implode, hydrodynamic liquid jets are supposed to be generated, which, when they strike the tooth surface, detach the biofilm with the aid of the high shear stresses generated.

The approaches introduced thus far struggle with two types of difficulties: on the one hand, the size of the generated vapor bubble, which also determines the cleaning intensity, is very difficult to control. On the other hand, the vapor bubbles are generated very close to an actuator (e.g., vibrating scaler), where they implode immediately due to their short lifespan, which minimizes the cleaning distance. This process in turn enables an application only locally in the professional tooth cleaning segment.

DE 20 2016 101 191 U1 has disclosed a brush head for an electric toothbrush, which is intended to surround the tooth on all sides and on which bristles are arranged for cleaning.

U.S. Pat. No. 3,401,690 A has disclosed a cleaning device in which ultrasound is applied to a surface via a liquid by means of a clamp, which fits over at least one tooth.

US 2005/0091770 A1 has disclosed a toothbrush, which works like a normal electric toothbrush, but in addition has an ultrasonic generator, which is supposed to introduce acoustic energy into a cleaning liquid.

US 2017/0189149 A1 has disclosed a system intended to whiten teeth with an ultrasonic device. For this purpose, a mouthpiece is provided which has a volume for the upper arch and a volume for the lower arch, wherein ultrasonic generators are positioned in the mouthpiece facing the teeth, which can apply ultrasonic energy to the tooth surface.

This is supposed to produce an effect known as ultrasound streaming; it is explained in this context that the temperature must be controlled and also that bubble formation must be prevented since these hinder the transmission of the ultrasound. A frequency of 20 KHz to 100 kHz is supposed to be used; in this case, cavitation is to be selectively induced so that vapor bubbles form which implode on the surface of the tooth, with the intent being to generate local temperatures of up to 5000 Kelvin and local pressures of up to 1000 atmospheres.

The disadvantage here is that the energies introduced are so high that damage to the tissue is practically inevitable.

WO 2007/060644 A2 has disclosed a method and device for removing biofilm by means of so-called microstreaming. In this case, gas bubbles are supposed to be set into resonance by ultrasound, which is supposed to produce a cleaning effect. The purpose of the ultrasonic excitation is to cause the gas bubbles to vibrate, which induces an acoustic flow in a small area in the vicinity of the bubble. This acoustic flow is also known as “microstreaming.” This micro-streaming is said to generate shear forces capable of removing the biofilm. The corresponding gas bubbles can be performed and, in particular, these bubbles can also be generated in a phospholipid or protein environment to stabilize them.

WO 2009/077291 A2 also describes a method of introducing antimicrobial reagents into a biofilm; in this case, gas bubbles are introduced into a treatment chamber inside a plastic envelope, the plastic envelope is then destroyed by ultrasound and the bubbles are thus released. The gas bubbles, in turn, are excited by the ultrasound frequency in such a way that they vibrate and, after reaching a maximum amplitude of vibration, collapse, thereby rupturing the biofilm.

WO 2010/076705 A1 has disclosed a toothbrush, which, in addition to bristles, includes an ultrasonic generator that introduces ultrasound into a treatment chamber and microbubbles are also introduced. This can, but does not have to, generate cavitation.

WO 2020/212214 A1 discloses a method in which a toothbrush is to be coupled to a water jet device, the water jet device being controlled in such a way that when the toothbrush is guided past the interdental areas, a water jet rinses the interdental areas. Suitable acceleration, speed, or displacement sensors are to be used for this purpose.

WO 2020/212248 A1 discloses a method in which a water jet device is also coupled to a toothbrush, wherein a control device is present, which makes an assumption as to where the cleaning device is located in the mouth, using predetermined data and user-specific data, wherein among other things, the data includes data relating to the cleaning activity of the user or the operation of the cleaning device and are used to make an assumption as to the location in order to rinse an interdental area with the water jet when the area is reached.

A disadvantage of the known methods is that experiments have shown that cleaning with ultrasonically generated (imploding) bubbles alone is not sufficient. Either the cleaning power is too low or the cleaning power is higher, but at a higher cleaning power, which is nowhere near sufficient, an energy range is reached which is not safe because in these energy ranges, cavitation can occur, which can lead to destruction in some places in both the gums and the tooth material. To prevent such destruction, this range must be avoided over a fairly wide area, which renders the cleaning performance ineffective. Combining microbubbles with conventional toothbrushes ultimately only combines the disadvantages of both technologies.

In addition, it has been determined that a cleaning of all surfaces to be cleaned, i.e. also the interdental spaces, does not take place reliably with the bubbles generated by ultrasound.

In general, existing methods for cleaning surfaces and in particular, surfaces in the oral cavity of mammals and humans are complicated, error-prone, and often be implemented without long-term damage to the teeth.

### SUMMARY OF THE INVENTION

The object of the invention is to create a method for cleaning surfaces, and in particular tooth and gum surfaces and interdental spaces, which removes biofilm from gums, interdental spaces, and teeth simply, quickly, reliably and also effectively and safely.

The object is attained with the features described and claimed herein.

Advantageous modifications are described and claimed herein.

Another object is to create a treatment solution that can be used to clean surfaces easily and effectively.

This object is attained with a treatment solution described and claimed herein.

Advantageous modifications are described and claimed herein.

Another object is to use the above-described treatment solution to achieve a simple and effective surface cleaning.

This object is attained with a treatment solution described and claimed herein.

Another object is to use the above-described method for cleaning surfaces and in particular tooth and gum surfaces and interdental spaces, in a way that removes biofilm from gums, interdental spaces, and teeth simply, quickly, reliably and also effectively and safely.

This object is attained with a treatment solution described and claimed herein.

According to the invention, a completely new approach is taken in cleaning surfaces and, in particular, surfaces in the oral cavity, which enables cleaning and in particular, selective cleaning of biofilm from surfaces.

According to the invention, a cleaning solution consisting of two liquid components is used.

The cleaning of the surface and especially in the mouth is triggered by an exothermic chemical reaction, hereinafter referred to as ignition, of a medium, hereinafter referred to as the ignition medium, within a liquid medium, in a volume that is adjacent to the surface to be cleaned, hereinafter referred to as the cleaning surface. The ignition volume is advantageously located close to the cleaning surface and can also touch it. As a rule, a large number of ignition volumes are located in the cleaning volume at the same time.

In a first phase, the ignition of the ignition medium causes an expansion and in a second phase, causes a collapse of the ignition volume in the cleaning liquid, whereby pressure forces and their gradients, as well as shear forces act on the cleaning surface. Particles, hereinafter referred to as cleaning particles, locally intensify the forces of the cleaning fluid or cleaning liquid since the latter acts on the cleaning particles and these locally transmit these forces to a small partial surface of the cleaning surface that they are in direct physical contact with, whereby which loosens and removes contaminants. In the second phase (collapse), the formation of an intense concentrated liquid jet, which generates particularly high local shear rates or shear stresses, can also produce a particularly powerful local cleaning effect.

Preferably, the ignition medium is a gas mixture and preferably, for example, a hydrogen-oxygen mixture, referred to hereinafter as oxyhydrogen.

Preferably, the ignition medium is finely dispersed in a treatment solution in the form of small gas bubbles.

In the field of contactless cleaning, the cleaning effect of imploding or collapsing vapor bubbles has been discussed in recent years. Such vapor bubbles have been obtained either through the application of ultrasound, through spot heating by laser, or through hydrodynamic cavitation. When the vapor bubbles implode, hydrodynamic liquid jets are generated, which, when they strike the tooth surface, detach the biofilm with the aid of the high shear stresses generated. The effective area and intensity depend heavily on the size of the vapor bubble and the distance of the vapor bubble from the surface.

The approaches introduced thus far struggle with two types of difficulties: on the one hand, the size of the generated vapor bubble, which also determines the cleaning intensity, is very difficult to control. On the other hand, the vapor bubbles are generated very close to an actuator (e.g., vibrating scaler), where they implode immediately due to their short lifespan, which minimizes the cleaning distance. This process in turn enables an application only locally in the professional tooth cleaning segment.

In addition, the solutions described above are all very energy-intensive, making it extremely difficult to use them in a rechargeable or battery-powered device.

The invention uses the effect of expanding and collapsing bubbles, but makes it much simpler and more effective by using gas bubbles containing at least two gaseous components that can react chemically with each other, specifically with expansion of the bubble, collapse of the bubble, or the two effects in succession.

An advantage of using oxyhydrogen is that its combustion produces water, resulting in very little or no undesirable chemical by-products in the cleaning liquid.

The invention also successfully achieves a selective cleaning of surfaces covered with biofilm or, in general, of a layer to be cleaned such as the teeth, interdental spaces, gums, cheeks, palate or tongue when a suitable agent is used.

The advantage of this is that the cleaning intensity can be adapted to the corresponding contaminated area; this is particularly advantageous because it allows optimization of cleaning time and energy consumption and also because the intensity can be adapted to the medically tolerable limit values of the respective area.

According to the invention, this is successfully achieved by bringing a first component of the cleaning solution into contact with the surface to be cleaned and thereby achieving a coupling of a first active substance to the biofilm. The first active ingredient in this case is for example a catalytically active substance that causes a chemical reaction of the at least two gaseous components.

Then a second component of the treatment solution is brought into contact with the surface to be cleaned, with the second component containing the ignition medium distributed in small ignition volumes, in particular finely distributed bubbles.

On the one hand, the micro-movement of the second component of the treatment solution is then used for cleaning the surface, which, triggered by the for example exothermic chemical reaction, generates expansion of the sealed volume, namely the bubble referred to hereinafter as the ignition volume, and optionally, the subsequent collapse thereof.

To remove the detached biofilm and prevent reattachment, it is sufficient if the second component is moved along the surface to be cleaned in order to generate corresponding currents. For example, the second component of the treatment solution can be moved like an oral rinse in the oral cavity.

After a predetermined treatment time, the treatment solution can be removed. When used in the oral cavity, the treatment solution can be spit out.

To assist with the cleaning by the gas bubbles, the second component of the treatment solution can contain particles that interact with the microflows.

On the one hand, these micro-movements of the second component of the treatment solution generate a shear velocity in the treatment solution, which generates the shear forces directly at the cleaning surface, and on the other hand, the movement of the fluid causes the particles to experience forces at the cleaning surface, which generate pressure and shear stresses at the cleaning surface due to direct contact with the cleaning surface. In addition, pressure gradients in the cleaning fluid due to acceleration forces can contribute to the cleaning, particularly when the collapse of the ignition volume results in the formation of a jet directed at the cleaning surface.

The treatment solution or more specifically, the first and/or the second component of the treatment solution, can be a calibrated treatment solution and on the one hand, can be ordinary water, but a special liquid can also be used, which is adapted to the cleaning parameters depending on the special composition with alcohols etc., through a corresponding thickening by means of thickeners or through the addition of cleaning enhancers (particles, cellulose fibers, etc.), flavorings, and other components.

The cleaning effect of the igniting gas bubble depends to a large extent on the dynamics of the collapse. To achieve cleaning of dental plaque, the time required for the vapor bubble to collapse should be in the range of 0.01 ms to 2 ms, in particular 0.1 to 0.5 ms, and more precisely in a time between 30 us to 300 μs. This applies to bubble sizes with a diameter of 50 μm to 500 μm. The Minnaert resonance frequency (for ideal gas) is

$$f = \frac{1}{2 π \text{?}} {( \frac{3 \text{?} {p}_{A}}{ρ} )}^{1 / 2}$$

$$\text{?} \text{indicates text missing or illegible when filed}$$

When using particles, particles from 1 μm to 0.5 mm can be used.

The desired particle density is preferably less than 30 volume percent, in particular less than 20 volume percent, especially less than 15 volume percent, further preferably less than 10 volume percent, and in particular less than 5 volume percent, in each case based on the volume of the treatment solution used.

The invention therefore relates to a method for cleaning surfaces, wherein the surface to be cleaned is simultaneously or successively brought into contact with two components of a treatment solution, wherein a first component of the treatment solution has a first active ingredient that is embodied to couple to a contaminant to be cleaned off and is embodied to stimulate or catalyze a chemical reaction of a second active ingredient in a second component of the treatment solution, wherein the second active ingredient used in the second component of the treatment solution is a gas mixture having at least two gaseous components that react chemically with each other under the influence of the first active ingredient.

According to one modification, the contaminant to be cleaned off is a biofilm.

According to one modification, oxyhydrogen is used as a second active ingredient.

According to one modification, a composition containing platinum or carbon palladium is used as the first active ingredient.

According to one modification, a first active ingredient is used, which is hydrophobized.

According to one modification, the first active ingredient comprises a coating that contains one or several or all of the following group: carbon, silica, silica aerogels, hydrophobic or hydrophobized nanomaterial, carbon aerogels, titanium dioxide, titanium dioxide aerogels, silanes, gold, lipids, peptides, amino acids, and proteins.

According to one modification, the first and/or a second treatment solution contains, in addition to the first and/or second active ingredient, one or several or all of the following group: water, monohydric or polyhydric alcohols, thickeners, optical brighteners, fluorescein, natural and artificial flavors, stabilizers, acid buffers, alkali buffers, antioxidants, detergent enhancers, particles, and cellulose fibers.

According to one modification, the second component of the treatment solution contains 0.1-5 vol % particles.

According to one modification, mineral particles or cellulose-based particles are used for the particles.

According to one modification, particles with a particle size of 5 μm to 500 μm, in particular 20-120 μm, are used.

According to one modification, the particle density in the second component is below 30 volume percent, in particular below 20 volume percent, and especially below 15 volume percent relative to the liquid contained in the sealed volume.

According to one modification, the second active ingredient is selected and the bubble size is dimensioned such that the time required for the bubble to collapse is in the range from 0.01 ms to 2 ms, in particular 0.1 to 0.5 ms, more precisely between 30 us and 300 μs, for bubble sizes with a diameter of 50 μm to 500 μm.

Another aspect of the invention relates to a treatment solution for cleaning surfaces, wherein the treatment solution has two components, wherein a first component of the treatment solution has a first active ingredient that is embodied to couple to a contaminant to be cleaned off and is embodied to stimulate or catalyze a chemical reaction of a second active ingredient in a second component of the treatment solution, wherein the second active ingredient used in the second component of the treatment solution is a gas mixture having at least two gaseous components that react chemically with each other under the influence of the first active ingredient.

According to one modification, the second active ingredient is present in the form of finely dispersed nanobubbles with diameters of 20 nm to 200 nm, in particular 75 nm to 125 nm, especially 100 nm, or microbubbles with diameters of 5 μm to 2000 μm, in particular 50-500 μm.

According to one modification, the second active ingredient is oxyhydrogen.

According to one modification, the first active ingredient comprises one or several or all of the following group: platinum, carbon, palladium, titanium, titanium dioxide, zirconium, and zirconium dioxide.

According to one modification, the first component and/or the second component of the treatment solution contains, in addition to the first and/or second active ingredient, one or several or all of the following group: water, monohydric or polyhydric alcohols, thickeners, optical brighteners, fluorescein, natural and artificial flavors, stabilizers, acid buffers, alkali buffers, antioxidants, detergent enhancers, particles, and cellulose fibers.

The invention also relates to the use of the treatment solution for cleaning surfaces in the oral cavity.

The invention also relates to the use of the method for cleaning surfaces in the oral cavity.

### BRIEF DESCRIPTION OF THE DRAWINGS

The invention will be explained by way of example below based on the drawings. In the drawings:

FIG. 1: is a highly schematic depiction of the action of the method according to the invention on a surface in the oral cavity;

FIG. 2: shows the action of a selective cleaning according to the invention;

FIG. 3: shows the recombination of bubbles;

FIG. 4: shows microbubbles formed from nanobubbles and liquid, before and after the ignition

FIG. 5: is a highly schematic depiction of the coating of catalyst particles;

FIG. 6: shows the catalytic ignition of oxyhydrogen microbubbles on catalyst particles in an experiment;

### DETAILED DESCRIPTION OF THE INVENTION

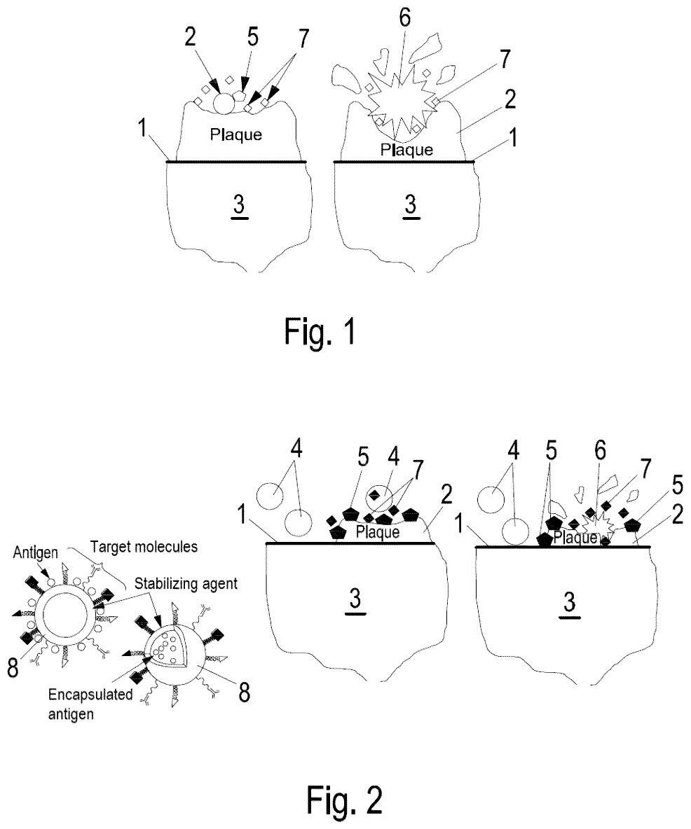

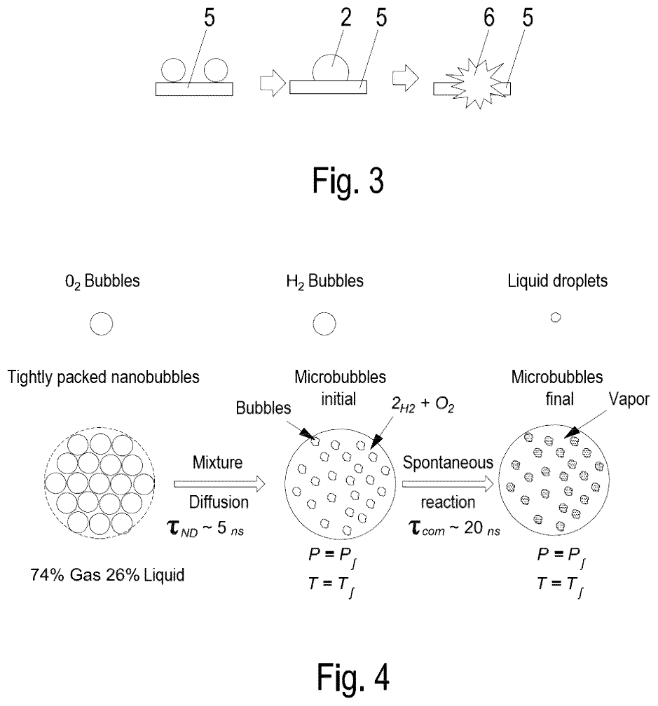

FIG. 1 is a highly schematic depiction of basic procedures of the method.

The method is basically suitable for all surfaces 1 from which a comparatively soft coating 2 is to be removed. FIG. 1 shows a tooth 3 as the base surface and plaque, i.e. biofilm, as the coating 2. In principle, the base surface could also be a metal, for example a stainless steel, and the coating, for example also biofilm, or coatings of the kind that can occur as contamination of surgical instruments and the like.

It is clear how a micro-gas bubble 4 attaches to the biofilm surface, simultaneously coming into contact with a catalytic particle 5. Contact with the catalytic particle 5 results in an ignition 6 and a reaction of the components inside the micro-gas bubble 4. In addition, cleaning particles 7 can be present, which can intensify existing shear forces.

In this case, the components of the micro-gas bubble 4 are hydrogen and oxygen, which together can undergo an oxyhydrogen reaction. This is shown on the right-hand side in FIG. 1. This results in an expansion and subsequent collapse so that the expansion and subsequent collapse exert different shear forces on the biofilm, causing it to be detached or blown off.

The bubbles 2 in this case can be relatively small and in particular have a diameter of 10-30 μm.

The catalytic particles 5 can be platinum, for example, but also any other catalytic particles that can be catalytically active for the corresponding pairing of reactive gases.

In the oxyhydrogen reaction, the ratio of hydrogen and oxygen influences the strength of the reaction.

Preferably, therefore, a stoichiometric initial concentration is sought.

FIG. 2 shows how a selective cleaning according to the invention can be carried out. According to the invention, the catalytic particles 5 are modified so that they dock with or adhere to the coating 2 to be cleaned off, for example a biofilm 2. In particular, they can be modified so that they to the bacteria 8 forming the biofilm.

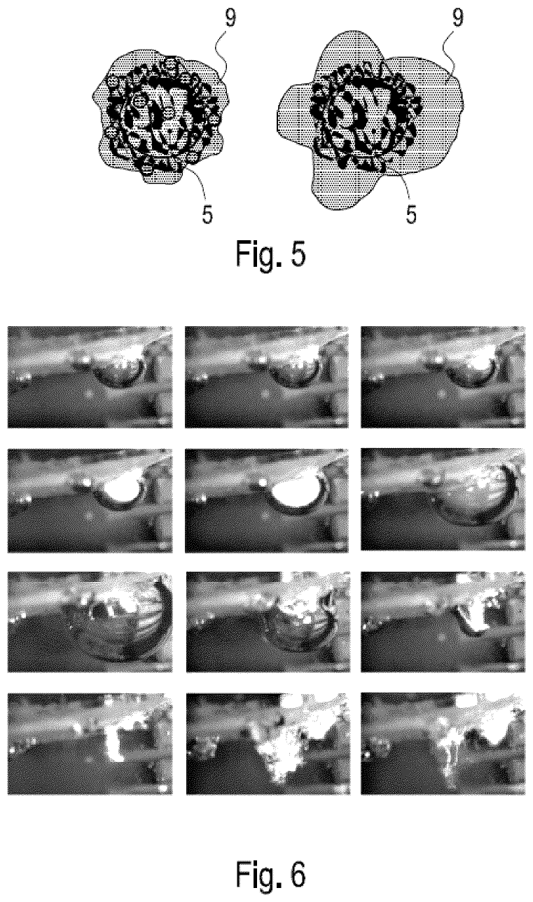

Depending on the biofilm 2, this can be done, for example, by providing the catalytic particles with a sheath 9 (FIG. 5), which docks with or adheres to the bacteria 8 forming the biofilm.

These are, for example, lipid coatings, peptide groups, or peptide sheaths. In this case, the correspondingly modified or prepared catalytic particles 5 can be applied to the surface 1 to be cleaned before the application of the gas bubbles 2, in particular oxyhydrogen bubbles.

Preferably, the modification of the catalytic particles 5 is such that they only dock with or adhere to the coating 2 and not “clean” areas of the surface 1. This ensures more effective utilization of the bubbles 2 on the one hand and a time-efficient cleaning on the other and also protects areas that are not covered by the coating 2 since no reactions take place there.

The catalyst and, in particular, the correspondingly coated catalyst particles 5 can render even very small bubbles 2 ignitable by reducing the activation energy, which also means that there is no release of extremely high temperatures, thus causing only very low thermal or chemical stresses.

The catalyst for igniting the gas mixture inside the gas bubble or the ignition medium can be a photocatalyst, which is brought into contact with the ignition medium, especially if the ignition volume is particularly small. A prerequisite for this is that a coating, which enables adhesion to the biofilm, is provided to the catalyst preferably not over the entire surface, but only on part of it so that catalyst surfaces remain available for contact with the bubbles 2 or the ignition medium.

Alternatively, the catalyst particles 5 can be coated over their entire surface, but the coating dissolves after a short time in the areas where no adhesion to the biofilm has taken place. Consequently, the application with microbubbles 2 occurs only after a short waiting period.

By advantageously coating the catalyst in such a way that it has a high affinity for adhering to the biofilm, an advantageous selectivity of the cleaning can be achieved because the cleaning is carried out substantially on the biofilm and hardly at all in “clean” areas. The above-described catalyst was described as being in particulate form, but this does not exclude the possibility of the catalyst also being in liquid or gaseous form. For example, it is not out of the question for the cleaning fluid itself to be the catalyst if the oxyhydrogen is present in the form of so-called nanobubbles.

In this case, the catalyst solves the problem that a self-sustaining exothermic chemical reaction would not be possible when the ignition volumes of bubbles 2 are very small because over the surface of the ignition volume, too much thermal energy would be lost to permit this.

It is also possible to produce the gas bubbles 2 in the form of nanobubbles with which a spontaneous ignition cannot occur, wherein when applied to the surface or when in the second component, these nanobubbles combine due to surface effects to form a microbubble, which then spontaneously ignites or can be ignited at the surface.

In this case, the second component can also contain cleaning particles 7. But this does not exclude the possibility of these particles 7 also having catalytic properties or conversely being comprised or partially comprised of catalyst particles. These nanobubbles are combined at very high density inside the cleaning volume to form microbubbles 2 and can then induce the above-described effects through spontaneous ignition.

This effect, which has already been scientifically studied, is also shown in FIG. 4, in which nanobubbles containing 74% different gases and 26% liquid diffuse and combine to form microbubbles, which can react spontaneously and form a final microbubble consisting of vapor, which can subsequently collapse.

Preferably, the catalyst is modified so that it has an affinity for the coating to be cleaned so that it adheres particularly well for example to the biofilm in the example in which cleaning is carried out in the oral cavity. This can be achieved by means of the organic coating of the catalyst particles mentioned above, but inorganic particle coatings such as gold can also be used.

Due to the affinity of the catalyst for adhering, for example, more strongly or only to the biofilm, the ignitions mostly occur close to the biofilm-covered surface to be cleaned, resulting in a higher efficiency of the cleaning performance as well as selectivity and thus a sparing of the rest of the surface and lower consumption of the ignition medium, i.e. the bubbles.

Possible coatings for the catalysts are shown in FIG. 6, for example, wherein the catalysts can have a first coating of carbon, carbon aerogels, silica, or silica aerogels, wherein hydrophobic or hydrophobized nanomaterials such as silanes can be present. A second coating can consist of hydrophilic coatings such as SIO₂ or gold and the like, wherein without a hydrophilic coating, a large hydrophobic space can be present, which facilitates coalescence. In this case, care must be taken not to have too large a hydrophobic space combined with a high catalyst particle density since this can cause large contiguous hydrophobic areas to form on the tooth surface. This is disadvantageous because it causes the oxyhydrogen to spread over the surface and not ignite locally. This must be combatted through partial hydrophilization and reduction of the hydrophobic areas in order to prevent the hydrophobic areas or catalyst particles from sticking together, which in turn allows a higher catalyst particle density to be achieved. The catalysts can be platinum, palladium, or carbon, and photocatalysts such as titanium oxide or titanium oxide aerogel, carbon, and others can also be provided.

The general properties that a catalyst should have for use in the method of the invention are good thermal insulation, high surface area, a hydrophobic space for fast reaction kinetics, good catalytic properties for a fast reaction, biocompatibility, low heat capacity, and preferably high affinity for docking with the material to be cleaned, for example biofilm.

In this connection, it has turned out in particular that a certain hydrophobizing of the catalytic particles and in particular of platinum particles is advantageous. This permits a good ignition of the ignition medium and in particular of oxyhydrogen bubbles at the water surface and under water, wherein a hydrophobic space, i.e. a layer of air on the surface of the catalyst, allows much faster reaction kinetics than if the platinum were covered with water.

When using oxyhydrogen bubbles, these can be produced from the gas mixture of hydrogen and oxygen through various methods. On the one hand, they can be added to the liquid in a Venturi nozzle; on the other hand, they can be generated in a so-called atomizer pump or also by ultrasound.

Electrolysis is naturally also possible and methods can be used in which microbubbles and nanobubbles are generated by supersaturation or by pressing through porous media or through miniscule holes.

Bubbles can also be obtained at the cleaning surface by means of a cleaning liquid that is supersaturated with oxyhydrogen. This can be done on the one hand by means of a generated pressure drop during the inflow, by heating the cleaning fluid, or by ultrasound.

The cleaning liquid can be ordinary water, but a liquid especially adapted to the bubbles and the cleaning can also be used, which is adapted to the cleaning parameters depending on the special composition with alcohols etc., through a corresponding thickening by means of thickeners or through the addition of cleaning enhancers (particles, cellulose fibers, etc.) and through its degree of degassing.

In order to bring the bubbles with the gas mixture as well as potential cleaning particles close to the surface to be cleaned, the liquid can be moved intensively in the oral cavity in the manner of an oral rinse.

The overall cleaning intensity is determined primarily by the size of the gas bubbles, the degree of degassing of the liquid, the viscosity of the liquid, the influence of particles or fibers incorporated into the liquid, and by the selected ignition method. In particular, the choice of a suitable catalyst, e.g. platinum or titanium dioxide, and a suitable coating of the catalyst to change its wettability in the cleaning liquid or its response to light each play a decisive role in this context.

FIG. 6 shows the successful ignition of a small gas bubble, its expansion as well as the implosion and the formation of a small jet.

The catalyst and cleaning fluid are applied without a handheld device through the use of gargling liquids. A catalyst liquid can be gargled first, with the catalyst adhering to the biofilm. Then the cleaning fluid, which contains the ignition medium, is gargled.

Optionally, the ignition medium and catalyst can be used in the same cleaning fluid.

Gargling liquids can be freshly prepared by a device, but a variant with capsules or other portion containers is also conceivable.

1-20. (canceled)

21. A method for cleaning a surface (1), comprising the steps of:
contacting the surface (1) to be cleaned simultaneously or successively with first and second components of a treatment solution;
the first component including a first active ingredient (5),
the second component including a second active ingredient;
the first active ingredient (5) being embodied to couple to a contaminant (2) to be cleaned off the surface (1) and to stimulate or catalyze a chemical reaction of the second active ingredient;
wherein the second active ingredient includes a gas mixture having at least two gaseous components that react chemically with each other under the influence of the first active ingredient.

22. The method of claim 21, wherein the contaminant (2) comprises a biofilm.

23. The method of claim 22, wherein the second active ingredient comprises oxyhydrogen.

24. The method of claim 21, wherein the first active ingredient (5) comprises a composition containing at least one of platinum and carbon palladium.

25. The method of claim 21, wherein the first active ingredient (5) is hydrophobized.

26. The method of claim 21, wherein the first component further comprises a thermally insulative coating (9) around the first active ingredient (5).

27. The method of claim 26, wherein the coating (9) comprises at least one of carbon, silica, silica aerogels, hydrophobic or hydrophobized nanomaterial, silanes, gold, lipids, peptides, amino acids, and proteins.

28. The method of claim 21, wherein at least one of the first and second treatment solutions further comprises at least one ingredient selected from water, monohydric alcohol, polyhydric alcohol, thickener, optical brightener, fluorescein, flavor ingredient, stabilizer, acid buffer, alkali buffer, antioxidant, detergent enhancer, particles, and cellulose fibers.

29. The method of claim 21, wherein the second component of the treatment solution further comprises 0.1-5% by volume cleaning particles (7).

30. The method of claim 29, wherein the cleaning particles (7) comprise at least one of mineral particles and cellulose-based particles.

31. The method of claim 29, wherein the cleaning particles (7) have a particle size of 5-500 μm.

32. The method of claim 21, wherein the second component has a particle density below about 30 volume percent.

33. The method according to claim 21, wherein the second active ingredient is provided in the form of bubbles configured and dimensioned so that a time required for the bubbles to collapse is about 0.01 ms to about 2 ms for bubbles having a diameter of 50 μm to 500 μm.

34. A treatment solution for cleaning surfaces, comprising a first component and a second component;
the first component including a first active ingredient (5);
the second component including a second active ingredient;
the first active ingredient (5) being embodied to couple to a contaminant (2) to be cleaned off a surface and to stimulate or catalyze a chemical reaction of the second active ingredient;
wherein the second active ingredient includes a gas mixture having at least two gaseous components that react chemically with each other under the influence of the first active ingredient (5).

35. The treatment solution of claim 34, wherein the second active ingredient is present in the form of finely dispersed nanobubbles having diameters of about 20 nm to about 200 nm, or microbubbles having diameters of about 5 μm to about 2000 μm.

36. The treatment solution of claim 34, wherein the second active ingredient comprises oxyhydrogen.

37. The treatment solution of claim 34, wherein the first active ingredient (5) comprises at least one of platinum, carbon, palladium, titanium, titanium dioxide, zirconium, and zirconium dioxide.

38. The treatment solution of claim 34, wherein at least one of the first component and the second component further comprises at least one ingredient selected from water, monohydric alcohol, polyhydric alcohol, thickener, optical brightener, fluorescein, flavor ingredient, stabilizer, acid buffer, alkali buffer, antioxidant, detergent enhancer, particles, and cellulose fibers.

39. A method of using a treatment solution to clean a surface (1), comprising the steps of:
providing a treatment solution having a first component and a second component; and
contacting the surface (1) to be cleaned simultaneously or successively with first and second components of a treatment solution;
the first component including a first active ingredient (5),
the second component including a second active ingredient;
the first active ingredient (5) being embodied to couple to a contaminant (2) to be cleaned off the surface (1) and to stimulate or catalyze a chemical reaction of the second active ingredient;
wherein the second active ingredient includes a gas mixture having at least two gaseous components that react chemically with each other under the influence of the first active ingredient.

40. The method of claim 39, wherein the surface (1) is present in an oral cavity.
